(19) United States
(12) Patent Application Publication
(10) Pub. No.: US 2004/0265820 A1
(43) Pub. Date: Dec. 30, 2004

(54) Method of determining susceptibility to prion disease

(76) Inventors: Graham S. Jackson, London (GB); John Collinge, London (GB)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/485,279

(22) PCT Filed: Jul. 26, 2002
(86) PCT No.: PCT/GB02/03423

(30) Foreign Application Priority Data
Aug. 8, 2001 (GB) 01193390

(51) Int. Cl. C12Q 1/68; A01K 67/027

(52) U.S. Cl. 435/6; 800/15

(57) ABSTRACT

This invention relates to a method of determining the susceptibility of a subject to prion disease comprising the steps of: (a) providing a sample from said subject and in determining the human leucocyte antigen specificity of said sample, wherein if said sample has DQ7 human leucocyte antigen specificity, this indicates that said subject has a decreased susceptibility to prion disease, and if said sample does not have DQ7 human leucocyte antigen specificity, this indicates that said subject has an increased susceptibility to prion disease.

### FIELD OF INVENTION

The present invention relates to a method. In particular, the present invention relates to a method for determining the susceptibility of a subject to prion disease.

### BACKGROUND TO THE INVENTION

A prion is a transmissible particle devoid of nucleic acid. The most notable prion diseases are Bovine Spongiform Encephalopathy (BSE), Scrapie of Sheep and Creutzfeldt-Jakob Disease (CJD) of humans. The most common manifestation of CJD is sporadic CJD (sCJD) which occurs spontaneously in individuals. Iatrogenic CJD (iCJD) is a disease that results from accidental infection. Familial CJD (fCJD) is a form of CJD that occurs rarely in families and is caused by mutations of the human PrP gene. ‘New valiant’ CJD (vCJD) of humans is a distinct strain type of CJD that is associated with a pattern of PrP glycoforms that are different from those found for other types of CJD. It has been suggested that BSE may have passed from cattle resulting in vCJD in humans.

Prions appear to be composed exclusively of a modified isoform of prion protein (PrP) called PrP^{Sc}. The normal cellular PrP (called PrP^{C}) is converted into PrP^{Sc} through a post-translational process. During this process, the structure of PrP^{C} is altered and is accompanied by changes in the physiochemical properties of PrP. The amino acid sequence of PrP^{Sc} is determined by that encoded by the PrP gene of the mammalian host in which it last replicated.

The epidemiology of prion diseases—such as vCJD—is still uncertain. Attwood (2001) Trends in Biotechnology 19:8:283 has reported that research has now confirmed that BSE is indeed likely to have caused vCJD through alimentary contamination. Approximately one million contaminated cattle are thought to have entered the human food chain and, consequently, a major future epidemic of vCJD is predicted. Beale (2001) J R Soc Med 94:207-209 has reported that the long incubation period from prion infection to prion disease makes experimental work slow and difficult.

It is desirable to predict prion susceptibility. Prion susceptibility is thought to be a multifactorial condition. Prior art attempts to predict prion susceptibility are few, and have largely focussed on sCJD and on fCJD. There is a need for method(s) of predicting prion susceptibility.

One prior art study has correlated vCJD disease with a PrP sequence polymorphism (PrP Met 129—see Collinge et al 1996 Lancet vol 348 p.56).

The present invention seeks to overcome problem(s) associated with the prior art.

### SUMMARY OF THE INVENTION

The present invention is based upon the surprising finding that Human Leucocyte Antigen (HLA) class II type DQ7 is associated with susceptibility to prion disease. Subjects having DQ7 have a lower susceptibility to prion disease, whilst subjects not having a DQ7 HLA type show higher susceptibility to prion disease.

Thus, it is disclosed herein that lack of DQ7 in a subject correlates with increased susceptibility to prion disease.

In a first aspect, the invention provides a method of determining the susceptibility of a subject to prion disease comprising the steps of providing a sample from said subject and determining the human leucocyte antigen (HLA) specificity of said sample, wherein if said sample has DQ7 human leucocyte antigen specificity, then said subject has a decreased susceptibility to prion disease; and if said sample does not have DQ7 human leucocyte antigen specificity, then said subject has an increased susceptibility to prion disease.

The sample may be any tissue or bodily fluid capable of being HLA typed.

‘Determining the HLA specificity’ means elucidating the HLA specificity or HLA type. This process is commonly referred to as ‘HLA typing’. The terms ‘HLA specificity’ and ‘HLA type’ are used interchangeably herein.

When HLA typing the sample (ie. determining the HLA specificity), enough information must be collected to determine whether the sample is DQ7 or is not DQ7. The term ‘DQ7’ refers to a well-known HLA type which is described in myriad basic immunology texts (for example see ‘Immunology’, 1996 4^{th} Edition, edited by Roitt, Brostoff and Male, published by Mosby Times Mirror International Publishers Limited; see also Bodmer et al., 1994 Tissue Antigens vol 44 pp 1-18.)

The sample may be HLA typed by any suitable means known to those skilled in the art. Such means include serological typing, nucleic acid based methods such as DNA sequencing, sequence specific oligonucleotides (SSO), or sequence specific primers PCR (PCR-SSP), or any other suitable method. HLA typing is discussed in more detail below. Preferably, the sample is HLA typed using nucleic acid based methods. Thus, in another aspect, the invention relates to a method as described above wherein the human leucocyte antigen specificity is determined by nucleic acid-based methods. Preferably the sample is HLA typed using PCR-SSP. Thus, in another aspect, the invention relates to a method as described above wherein the nucleic acid-based methods comprise sequence specific primer polymerase chain reaction (PCR-SSP).

In another aspect, the invention relates to a method as described above wherein the prion disease is vCJD.

In another aspect, the invention relates to a method as described above wherein the sample is or is derived from bodily fluid or tissue.

In another aspect, the invention relates to a method as described above wherein the bodily fluid or tissue is blood.

In another aspect, the invention relates to a method as described above wherein the sample is or is derived from nucleic acid extracted from the bodily fluid or tissue.

### DETAILED DESCRIPTION OF THE INVENTION

Prion

As used herein the term “prion” has its usual meaning in the art and refers to a proteinaceous infectious particle that lacks nucleic acid.

Prion diseases of animals and humans are characterised by deposition of an abnormal conformation of a host-encoded protein. PrP^{Sc}. Human prion diseases have inherited, sporadic and acquired forms. A variant of Creutzfeldt-Jakob-Disease (vCJD) was identified in 1996 and results from exposure to bovine spongiform encephalopathy (BSE) prion infected material. vCJD has a pathogenesis distinct from that of sporadic and other forms of CJD, with a marked accumulation of PrP^{Sc} in lymphoreticular tissues.

Prion diseases, or transmissible spongiform encephalopathies, are neurodegenerative conditions caused by novel infectious agents lacking nucleic acid. The emergence of two new and related prion diseases: BSE in cattle and vCJD in humans has stimulated intense research effort into the molecular basis of disease pathogenesis. That vCJD arose as a result of dietary or other exposure to the BSE agent is supported by molecular and biological strain tipping studies and it is possible that many individuals have been infected. The distinctive pathogenesis of vCJD may be related to the presumed oral route of infection or to a prion strain-specific effect. Involvement of the lymphoreticular system (LRS) is also seen in sheep scrapie and murine models of scrapie where follicular dendritic cells have been shown to accumulate PrP^{Sc} early in the incubation period, well before onset of neurological signs. Prion replication in the LRS may be a prerequisite for neuroinvasion following peripheral inoculation with low doses of infectivity.

Background teachings on prions have been presented by Victor A. McKusick et al. on http://www.ncbi.nlm.nih.gov/Omim. The following information concerning prions has been extracted from that source:

Mutations in the prion protein gene are associated with Gerstmann-Straussler disease (GSD), Creutzfeldt-Jakob disease (CJD), and familial fatal insomnia, and aberrant isoforms of the prion protein can act as an infectious agent in these disorders as well as in kuru and in scrapie in sheep.

Prusiner (1982, 19S7) suggested that prions represent a new class of infectious agent that lacks nucleic acid. (The term prion, which was devised by Prusiner (1982), comes from ‘protein infectious agent.’) The prion diseases are neurodegenerative conditions transmissible by inoculation or inherited as autosomal dominant disorders. Prusiner (1994) reviewed the pathogenesis of transmissible spongiform encephalopathies and noted that a protease-resistant isoform of the prion protein was important in the pathogenesis of these diseases. Mestel (1996) reviewed the evidence for and against—and the opinions for and against—the existence of infectious proteins.

Tagliavini et al. (1991) purified and characterized proteins extracted from amyloid plaque cores isolated from 2 patients of the Indiana kindred. They found that the major component of GSD amyloid was an 11-kD degradation product of PrP, whose N-terminus corresponded to the glycine residue at position 58 of the amino acid sequence deduced from the human PrP cDNA. In addition, amyloid fractions contained larger PrP fragments with apparently intact N termini and amyloid P components. Tagliavini et al. (1991) interpreted these findings as indicating that the disease process leads to proteolytic cleavage of PrP, generating an amyloidogenic peptide that polymerizes into insoluble fibrils. Since no mutations of the structural gene were found in the family, factors other than the primary structure of PrP may play a crucial role in the process of amyloid formation.

One interpretation has been that the prion is a sialoglycoprotein whose synthesis is stimulated by the infectious agent that is the primary cause of this disorder and Manuelidis et al. (1987) presented evidence suggesting that the PrP peptide is not the infection agent in CJD. Pablos-Mendez et al. (1993) reviewed the ‘tortuous history of prion diseases’ and suggested an alternative to the idea that prions are infections, namely, that they are cytotoxic metabolites.

The authors suggested that studies of the processing of the metabolite PrP and trials of agents that enhance the appearance of this protein would be useful ways to test their hypothesis. Their model predicted that substances capable of blocking the catabolism of PrP would lead to its accumulation. Increasing PrP synthesis in transgenic mice shortens the latency in experimental scrapie. The hypothesis of Pablos-Mendez et al. (1993) suggested an intracellular derailment of the degradative rather than the synthetic pathway of PrP.

Forloni et al. (1993) found that the PrP peptide 106-126 has a high intrinsic ability to polymerize into amyloid-like fibrils in vitro. They also showed that neuronal death results from chouronic exposure of primacy rat hippocampal cultures to micromolar concentrations of a peptide corresponding to this peptide. They suggested that the neurotoxic effect of the peptide involves an apoptotic mechanism.

It has been suggested that the infectious, pathogenic agent of the transmissible spongiform encephalopathies is a protease-resistant, insoluble form of the PrP protein that is derived posttranslationally from the normal, protease-sensitive PrP protein (Beyreuther and Masters, 1994). Kocisko et al. (1994) reported the conversion of normal PrP protein to the protease-resistant PrP protein in a cell-free system composed of purified constituents. This selective conversion from the normal to the pathogenic form of PrP required the presence of preexisting pathogenic PrP. The authors showed that the conversion did not require biosynthesis of new PrP protein, its amino-linked glycosylation, or the presence of its normal glicosylphosphatidylinositol anchor. This provided direct evidence that the pathogenic PrP protein can be formed from specific protein-protein interactions between it and the normal PrP protein.

Rivera et al. (1989) described a 13-year-old male with a severe progressive neurologic disorder whose karotype showed a pseudodicentric chouromosome resulting from a telomeric fusion 15p:20p. In lymphocytes the centromeric constriction of the abnormal chouromosome was always that of chouromosome 20, whereas in fibroblasts both centromeres were alternately constricted. The authors suggested that centromere inactivation results from a modified conformation of the functional DNA sequences preventing normal binding to centromere-specific proteins. They also postulated that the patient's disorder, reminiscent of a spongy glioneuronal dystrophy as seen in Creutzfeldt-Jakob disease, may be secondary to the presence of a mutation in the prion protein.

Collinge et al. (1990) suggested that ‘prion disease’, whether familial or sporadic, may prove to be a more appropriate diagnostic term. An Indiana kindred with GSD disease was reported by Farlow et al. (1989) and Ghetti et al. (1989). Using PrP gene analysis in genetic prediction carries potential problems arising out of uncertainty about penetrance and the complications of presymptomatic testing in any inherited late-onset neurodegenerative disorder. Collinge et al. (1991) concluded, however, that it had a role to play in improving genetic counseling for families with inherited prion diseases, allowing presymptomatic diagnosis or exclusion of CJD or GSD in persons at risk.

Gajdusek (1991) provided a chart of the PRNP mutations found to date: 5 different mutations causing single amino acid changes and 5 insertions of 5, 6, 7, 8, or 9 octapeptide repeats. He also provided a table of 18 different amino acid substitutions that have been identified in the transthyretin gene (TTR; 176300) resulting in amyloidosis and drew a parallel between the behavior of the 2 classes of disorders.

Schellenberg et al. (1991) sought the missense mutations at codons 102, 117, and 200 of the PRNP gene, as well as the PRNP insertion mutations, which are associated with CJD and GSSD, in 76 families with Alzheimer disease, 127 presumably sporadic cases of Alzheimer disease, 16 cases of Down syndrome, and 256 normal controls, none was positive for any of these mutations. Jendroska et al. (1994) used histoblot immunostaining in an attempt to detect pathologic prion protein in 90 cases of various movement disorders including idiopathic Parkinson disease (PD; 168600), multiple system atrophy, diffuse Lewy body disease (127750). Steele-Richardson-Olszewski syndrome (260540), corticobasal degeneration, and Pick disease (172700). No pathologic prion protein was identified in any of these brain specimens, although it was readily detected in 4 controls with Creutzfeldt-Jakob disease. Perry et al. (1995) used SSCP to screen for mutations at the prion locus in 82 Alzheimer disease patients from 54 families (including 30 familial cases), as well as in 39 age-matched controls. They found a 24-bp deletion around codon 68 which removed 1 of the 5 gly-pro rich octarepeats in 2 affected sibs and 1 offspring in a late-onset Alzheimer disease family. However, the other affected individuals within the same pedigree did not share this deletion, which was also detected in 3 age-matched controls in 6 unaffected members from a late-onset Alzheimer disease family. Another octarepeat deletion was detected in 3 other individuals from the same Alzheimer disease family, of whom 2 were affected. No other mutations were found. Perry et al. (1995) concluded that there was no evidence for association between prion protein mutations and Alzheimer disease in their survey.

Hsiao et al. (1990) found no mutation in the open reading frame of the PrP gene in 3 members of the family analyzed, but Hsiao et al. (1992) later demonstrated a phe198-to-ser mutation: see 176640.0011.

Palmer and Collinge (1993) reviewed mutations and polymorphisms in the prion protein gene.

Chapman et al. (1996) demonstrated fatal insomnia and significant thalamic pathology in a patient heterozygous for the pathogenic ])sine mutation at codon 200 (176640.0006) and homozygous for methionine at codon 129 of the prion protein gene. They stressed the similarity of this phenotype to that associated with mutations in codon 178 (176640.0010).

Collinge et al (1996) investigated a wide range of cases of human prion disease to identify patterns of protease-resistant PrP that might indicate different naturally occurring prion strain types. They studied protease resistant PrP from ‘new variant’ CJD to determine whether it represents a distinct strain type that can be differentiated by molecular criteria from other forms of CJD. Collinge et al. (1996, demonstrated that sporadic CJD and iatrogenic CJD (usually due to administration of growth hormone from cadaver brain) is associated with 3 distinct patterns of protease-resistant PrP on Western blots. Types 1 and 2 are seen in sporadic CJD and in some cases of iatrogenic CJD. A third tripe is seen in acquired prion diseases with a peripheral route of exposure to prions. Collinge et al.(1996) reported that ‘new variant’ CJD is associated with a unique and highly consisten appearance of protease-resistant PrP on Western blots involving a characteristic pattern of glycosylation of the PrP. Transmission of CJD to inbred mice produced a PrP pattern characteristic of the inoculated CJD. Transmission of bovine spongiform encephalopathy lo (BSE) prion produced a glycoform ratio pattern of PrP closely similar to that of ‘new variant’ CJD. They found that the PrP from experimental BSE in macaques and naturally acquired BSE in domestic cats showed a glycoform pattern indistinguishable from that of experimental murine BSE and ‘new variant’ CJD. The report of Collinge et al. (1996) was reviewed by Aguzzi and Weissmann (1996), who concluded that Collinge et al. (1996) had reviewed the neuropathologic and clinical features of the ‘new variant’ of CJD that was related to BSE.

Prusiner (1996) provided a comprehensive review of the molecular biology and genetics of prion diseases. Collinge (1997) likewise reviewed this topic. He recognized 3 categories of human prion diseases: (1) the acquired forms include kuru and iatrogenic CJD; (2) sporadic forms include CJD in typical and atypical forms; (3) inherited forms include familial CJD. Gerstmann-Straussler-Scheinker disease, fatal familial insomnia; and the various atypical dementias. Collinge (1997) tabulated 12 pathogenetic mutations that had been reported to that time. Noting that the ability of a protein to encode a disease phenotype represents a nonmendelian form of transmission important in biology. Collinge (1997) commented that it would be surprising if evolution had not used this method for other proteins in a range of species. He referred to the identification of prion-like mechanisms in yeast (Wickner, 1994; Ter Avanesyan et al. 1994).

Horwich and Weissman (1997) reviewed the central role of prion protein in the group of related transmissible neurodegenerative diseases. The data demonstrated that prion protein is required for the disease process, and that the conformational conversion of the prion protein from its normal soluble alpha-helical conformation to an insoluble beta-sheet state is intimately tied to the generation of disease and infectivity. They noted that much about the conversion process remains unclear.

Mallucci et al. (1999) described a large English family with autosomal dominant segregation of presenile dementia, ataxia, and other neuropsychiatric features. Diagnoses of demyelinating disease, Alzheimer disease, Creutzfeldt-Jakob disease, and Gerstmann-Straussler-Scheinker syndrome had been made in particular individuals at different times. Mallucci et al. (1999) also described an Irish family, likely to be part of the same kindred, in which diagnoses of multiple sclerosis, dementia, corticobasal degeneration, and ‘new variant’ CJD had been considered in affected individuals. Molecular studies identified the disorder as prion disease due to an ala117-to-val mutation in the PRNP gene. They emphasized the diversity of phenotypic expression seen in these kindreds and proposed that inherited prion disease should be excluded by PRNP analysis in any individual presenting with atypical presenile dementia or neuropsychiatric features and ataxia, including suspected cases of ‘new variant’ CJD. Hegde et al. (1999) demonstrated that transmissible and genetic prion diseases share a common pathway of neurodegeneration. Hegde et al. (1999) observed that the effectiveness of accumulated PrP^{Sc}, an abnormally folded isoform, in causing neurodegenerative disease depends upon the predilection of host-encoded PrP to be made in a transmembrane form, termed CtmPrP. Furthermore, the time course of PrP^{Sc} accumulation in transmissible prion disease is followed closely by increased generation of CtmPrP. Thus, the accumulation of PrP^{Sc} appears to modulate in trans the events involved in generating or metabolizing CtmPrP. Hegde et al. (1999) concluded that together these data suggested that the events of CtmPrP-mediated neurodegeneration may represent a common step in the pathogenesis of genetic and infection prion diseases.

PrP^{c}, the cellular, nonpathogenic isoform of PrP, is a ubiquitous glycoprotein expressed strongly in neurons. Mouillet-Richard et al. (2000) used the murine 1C11 neuronal differentiation model to search for PrP^{c}-dependent signal transduction thourough antibody-mediated crosslinking. The 1C11 clone is a committed neuroectodermal progenitor with an epithelial morphology that lacks neuron-associated functions. Upon induction, 1C11 cells develop a neural-like morphology, and may differentiate either into serotonergic or noradrenergic cells. The choice between the 2 differentiation pathways depends on the set of inducers used. Ligation of PrP^{c} with specific antibodies induced a marked decrease in the phosphorylation level of the tyrosine kinase FYN (137025) in both serotonergic and noradrenergic cells. The coupling of PrP^{c} to FYN was dependent upon caveolin-1 (601047). Mouillet-Richard et al. (2000) suggested that clathourin (see 118960) might also contribute to this coupling. The ability of the 1C11 cell line to trigger PrP^{c}-dependent FYN activation was restricted to its fully differentiated serotonergic or noradrenergic progenies. Moreover, the signaling activity of PrP^{c} occurred mainly at neurites. Mouillet-Richard et al. (2000) suggested that PrP^{c} may be a signal transduction protein.

Mapping

The human gene for prion-related protein has been mapped to 20p12-pter by a combination of somatic cell hybridization and in situ hybridization (Sparkes et al., 1986) and by spot blotting of DNA from sorted chouromosomes (Liao et al., 1986). Robakis et al. (1986) also assigned the PRNP locus to 20p by in situ hybridization.

By analysis of interstitial 20p deletions. Schnittger et al. (1992) demonstrated the following order of loci: pter—PRNP—SCG1 (118920)—BMP2A (112261)—PAX1 (167411)—cen. Puckett et al. (1991) identified 5-prime of the PRNP gene a RFLP that has a high degree of heterozygosity, which might serve as a useful marker for the pter-p12 region of chouromosome 20.

Riek et al. (1998) used the refined NMR structure of the mouse prion protein to investigate the structural basis of inherited human transmissible spongiform encephalopathies. In the cellular form of mouse prion protein, no spatial clustering of mutation sites was observed that would indicate the existence of disease-specific subdomains. A hydrogen bond between residues 128 and 178 provided a structural basis for the observed highly specific influence of a polymorphism at position 129 in human PRNP on the disease phenotype that segregates with the asp178-to-asn (D178N; 176640.0007) mutation. Overall, the NMR structure implied that only some of the disease-related amino acid replacements lead to reduced stability of the cellular form of PRNP, indicating that subtle structural differences in the mutant proteins may affect intermolecular signaling in a variety of different ways.

Windl et al. (1999) searched for mutations and polymorphisms in the coding region of the PRNP gene in 578 patients with suspect prion diseases referred to the German Creutzfeldt-Jakob disease surveillance unit over a period of 4.5 years. They found 40 cases with a missense mutation previously reported as pathogenic. Among these, the D178N mutation was the most common. In all of these cases, D178N was coupled with methionine at codon 129, resulting in the typical fatal familial insomnia genotype. Two novel missense mutations and several silent polymorphisms were found. In their FIG. 1, Windl et al. (1999) diagrammed the known pathogenic mutations in the coding region of PRNP.

History

Aguzzi and Brandner (1999) reviewed ‘the genetics of prions’ but raised the question of whether this is a contradiction in terms since the prion, which they defined as an enigmatic agent that causes transmissible spongiform encephalopathies, is a paradigm of nongenetic pathology. The protein-only hypothesis, originally put forward by Griffith (1967), says that prion infectivity is identical to scrapie protein, an abnormal form of the cellular protein, now referred to as PRNP. Replication occurs by the scrapie prion recruiting cellular prion and converting it into further scrapie prion. The newly formed scrapie prion will join the conversion cycle and lead to a chain reaction of events that results in an ever-faster accumulation of scrapie prion. This hypothesis gained widespread recognition and acceptance after Prusiner (1982) purified the pathologic protein and Weissmann and his colleagues (Oesch et al., 1985; Basler et al., 1986) cloned the gene that encodes the scrapie protein as well as its normal cellular counterpart PRNP. Even more momentum was achieved when Weissmann's group (Bueler et al., 1993) showed that genetic ablation of Prnp protects mice from experimental scrapie on exposure to prions, as predicted by the protein-only hypothesis. Aguzzi and Brandner (1999) considered the finding of linkage between familial forms of prion diseases and mutations in the prion gene to be an important landmark (Hsiao et al., 1989).

Animal Model

The structural gene for prion (Prn-p) has been mapped to mouse chouromosome 2. A second murine locus, Prn-i, which is closely linked to Prn-p, determines the length of the incubation period for scrapie in mice (Carlson et al., 1986). Yet another gene controlling scrapie incubation times, symbolized Pid-1, is located on mouse chouromosome 17. Scott et al. (1989) demonstrated that transgenic mice harboring the prion protein gene from the Syrian hamster, when inoculated with hamster scrapie prions, exhibited scrapie infectivity, incubation times, and prion protein amyloid plaques characteristic of the hamster. Hsiao et al. (1994) found that 2 lines of transgenic mice expressing high levels of the mutant P101L prion protein developed a neurologic illness and central nervous system pathology indistinguishable from experimental murine scrapie. Amino acid 102 in human prion protein corresponds to amino acid 101 in mouse prion protein, hence, the P101L murine mutation was the equivalent of the pro102-to-leu mutation (176640.0002) which causes Gerstmann-Straussler disease in the human. Hsiao et al. (1994) reported serial transmission of neurodegeneration to mice who expressed the P101L transgene at low levels and Syrian hamsters injected with brain extracts from the transgenic mice expressing high levels of mutant P101L prion protein. Although the high-expressing transgenic mice accumulated only low levels of infections prions in their brains, the serial transmission of disease to inoculated recipients argued that prion formation occurred de novo in the brains of these uninoculated animals and provided additional evidence that prions lack a foreign nucleic acid.

Studies on PrP knockout mice have been reported by Bueler et al. (1994), Manson et al. (1994), and Sakaguchi et al. (1996). Sakaguchi et al. (1996) reported that the PrP knockout mice produced by them were apparently normal until the age of 70 weeks, at which point they consistently began to show signs of cerebellar ataxia. Histologic studies revealed extensive loss of Purkinje cells in the majority of cerebellar folia. Atrophy of the cerebellum and dilatation of the fourth ventricle were noted. Similar pathologic changes were not noted in the PrP knockout mice produced by Bueler et al. (1994) and by Manson et al. (1994). Sakaguchi et al. (1996) noted that the difference in outcome may be due to strain differences or to differences in the extent of the knockout within the PrP gene. Notably, in all 3 lines of PrP knockout mice described, susceptibility to prion infection was lost.

Based on their studies in PrP null mice. Collinge et al. (1994) concluded that prion protein is necessary for normal synaptic function. They postulated that inherited prion disease may result from a dominant negative effect with generation of PrP^{Sc}, the posttranslationally modified form of cellular PrP, ultimately leading to progressive loss of functional PrP (PrP^{c}). Tobler et al. (1996) reported changes in circadian rhythm and sleep in PrP null mice and stressed that these alterations show; intriguing similarities with the sleep alterations in fatal familial insomnia.

Mice devoid of PrP develop normally but are resistant to scrapie; introduction of a PrP transgene restores susceptibility to the disease. To identify the regions of PrP necessary for this activity. Shmerling et al. (1998) prepared PrP knockout mice expressing PrPs with amino-proximal deletions. Surprisingly, PrP with deletion of residues 32-121 or 32-134, but not with shorter deletions, caused severe ataxia and neuronal death limited to the granular layer of the cerebellum as early as 1 to 3 months after birth. The defect was completely abolished by introducing 1 copy of a wildtype PrP gene. Shmerling et al. (1998) speculated that these truncated PrPs may be nonfunctional and compete with some other molecule with a PrP-like function for a common ligand.

Telling et al. (1996) reported observations that supported the view that the fundamental event in prion diseases is a conformational change in cellular prion protein whereby it is converted into the pathologic isoform PrP^{Sc}. They found that in fatal familial insomnia (FFI), the protease-resistant fragment of PrP^{Sc} after deglycosylation has a size of 19 kD, whereas that from other inherited and sporadic prion diseases is 21 kD. Extracts from the brains of FFI patients transmitted disease to transgenic mice expressing a chimeric human-mouse PrP gene about 200 days after inoculation and induced formation of the 19-kD PrP^{Sc} fragment, whereas extracts from the brains of familial and sporadic Creutzfeldt-Jakob disease patients produced the 21-kD PrP^{Sc} fragment in these mice. The results of Telling et al. (1996) indicated that the conformation of PrP^{Sc} functions as a template in directing the formation of nascent PrP^{Sc} and suggested a mechanism to explain strains of prions where diversity is encrypted in the conformation of PrP^{Sc}.

Lindquist (1997) pointed out that ‘some of the most exciting concepts in science issue from the unexpected collision of seemingly unrelated phenomena.’ The case in point she discussed was the suggestion by Wickner (1994) that 2 baffling problems in yeast genetics could be explained by an hypothesis similar to the prion hypothesis. Two yeast mutations provided a convincing case that the inheritance of phenotype can sometimes be based upon the inheritance of different protein conformations rather than upon the inheritance of different nucleic acids. Thus, yeast may provide important new tools for the study of prion-like processes. Furthermore, she suggested that prions need not be pathogenic. Indeed, she suggested that self-promoted structural changes in macromolecules lie at the heart of a wide variety of normal biologic processes, not only epigenetic phenomena, such as those associated with altered chouromatin structures, but also some normal, developmentally regulated events.

Hegde et al. (1998) studied the role of different topologic forms of PrP in transgenic mice expressing PrP mutations that alter the relative ratios of the topologic forms. One form is fully translocated into the ER lumen and is termed PrP-Sec. Two other forms span the ER membrane with orientation of either the carboxy-terminal to the lumen (PrP-Ctm) or the amino-terminal to the lumen (PrP-Ntm). F2-generation mice harboring mutations that resulted in high levels of PrP-Ctm showed onset of neurodegeneration at 58+/−11 days. Overexpression of PrP was not the cause. Neuropathology showed changes similar to those found in scrapie, but without the presence of PrP^{Sc}. The level of expression of PrP-Ctm correlated with severity of disease.

Supattapone et al. (1999) reported that expression of a redacted PrP of 106 amino acids with 2 large deletions in transgenic (Tg) mice deficient for wildtype PrP (Prnp −/−) supported prion propagation. Rocky Mountain laboratory (RML) prions containing full-length PrP^{Sc} produced disease in Tg(PrP106)Prnp −/− mice after approximately 300 days, while transmission of RML106 prions containing PrP^{Sc106} created disease in Tg(PrP106)Prnp −/− mice after approximately 66 days on repeated passage. This artificial transmission barrier for the passage of RML prions was diminished by the coexpression of wildtype mouse PrP^{c} in Tg(PrP106)Prnp +/− mice that developed scrapie in approximately 165 days, suggesting that wildtype mouse PrP acts in trans to accelerate replication of RML106 prions. Purified PrP^{Sc106} was protease resistant, formed filaments, and was insoluble in nondenaturing detergents.

Kuwahara et al. (1999) established hippocampal cell lines from Prnp −/− and Prnp +/+ mice. The cultures were established from 14-day-old mouse embryos. All 6 cell lines studied belonged to the neuronal precursor cell lineage, although they varied in their developmental stages. Kuwahara et al. (1999) found that serum removal from the cell culture caused apoptosis in the Prnp −/− cells but not in Prnp +/− cells. Transduction of the prion protein or the BCL2 gene suppressed apoptosis in Prnp −/− cells under serum-free conditions. Prnp −/− cells extended shorter neurites that Prnp +/− cells, but expression of PrP increased their length. Kuwahara et al. (1999) concluded that these findings supported the idea that the loss of function of wildtype prion protein may partly underlie the pathogenesis of prion diseases. The authors were prompted to try transduction of the BCL2 gene because BCL2 had previously been shown to interact with prion protein in a yeast 2-hybrid system. Their results suggested some interaction between BCL2 and PrP in mammalian cells as well.

In scrapie-infected mice, prions are found associated with splenic but not circulating B and T lymphocytes and in the stroma, which contains follicular dendritic cells. Formation and maintenance of mature follicular dendritic cells require the presence of B cells expressing membrane-bound lymphotoxin-alpha/beta. Treatment of mice with soluble lymphotoxin-beta receptor results in the disappearance of mature follicular dendritic cells from the spleen. Montrasio et al. (2000) demonstrated that this treatment abolished splenic prion accumulation and retards neuroinvasion after intraperitoneal scrapie inoculation. Montrasio et al. (2000) concluded that their data provided evidence that follicular dendritic cells are the principal sites for prion replication in the spleen.

Chiesa et al. (1998) generated lines of transgenic mice that expressed a mutant prion protein containing 14 octapeptide repeats, the human homolog of which is associated with an inherited prion dementia. This insertion was the largest identified to that time in the PRNP gene and was associated with a prion disease characterized by progressive dementia and ataxia, and by the presence of PrP-containing amyloid plaques in the cerebellum and basal ganglia (Owen et al., 1992; Duchen et al., 1993: Krasemann et al., 1995). Mice expressing the mutant protein developed a neurologic illness with prominent ataxia at 65 or 240 days of age, depending on whether the transgene array was, respectively, homozygous or hemizygous. Starting from birth, mutant PrP was converted into a protease-resistant and detergent-insoluble form that resembled the scrapie isoform of PrP, and this form accumulated dramatically in many brain regions thouroughout the lifetime of the mice. As PrP accumulated, there was massive apoptosis of granule cells in the cerebellum.

Major Histocompatability Complex

As used herein, the term “major histocompatibility complex” (MHC), has its usual meaning in the art and refers to a set of genes that encode a family of cellular antigens that function as “identity markers” on the surface of various cells. T-lymphocytes interact with these cells via their own receptors to carry out their adaptive immunological functions.

MHC is divided in to three classes of molecules (I, II and III) encoded within the murine and human MHCs. Class I and II molecules represent distinct structural entities. Class III MHC contains a diverse collection of over 20 genes with no established functional or structural similarities.

Background teachings on MHC Class II molecules have been presented by Victor A. McKusick et al on http://www.ncbi.nlm.nih.gov/Omim. The following information concerning MHC Class III molecules has been extracted from that source:

The genes for the heteromeric major histocompatibility complex class II proteins, the alpha and beta subunits, are clustered in the 6p21.3 region. Todd et al. (1987) Nature 329: 599-604 presented a map of the class II loci. They suggested that the structure of the DQ molecule, in particular residue 57 of the beta-chain, specifies the autoimmune response against insulin-producing islet cells that leads to insulin-dependent diabetes mellitus. Of the approximately 14 class II HLA genes within the HLA-D region, the DQ3.2-beta gene accounts for the well-documented association of HLA-DR4 with insulin dependent diabetes mellitus and is the single allele most highly correlated with this disease. Kwok et al. (1989) Proc. Nat. Acad. Sci. 86: 1027-1030 found that amino acid 45 was critical for generating serologic epitopes characterizing the DQ3.2-beta gene and its nondiabetic allele, DQ3.1-beta. Todd et al (1990) Proc. Nat. Acad. Sci. 87: 1094-1098 found that in Japanese IDDM was more strongly associated with HLA-DQ that with HLA-DR: that the A3 allele at the DQA1 locus was most strongly associated with disease; that the DQw8 allele of the DQB1 locus, which is associated with susceptibility to type J diabetes in Caucasians and blacks, was not increased in frequency in Japanese patients and that asp57-encoding DQB1 alleles, which are associated with reduced susceptibility to type I diabetes in Caucasians, was present in all except 1 of 49 Japanese patients and in all of 31 controls, in at least heterozygous state. Forty percent of patients were homozygous for asp57-encoding DQB1 alleles versus 35% of controls. The high frequency of asp57-encoding DQB1 alleles in Japanese may account for the rarity of type I diabetes in Japan.

The extremely high polymorphism of HLA class II transmembrane heterodimers is due to a few hypervariable segments present in the most external domain of their alpha and beta chains. Some changes in amino acid sequence are critical in disease susceptibility associations as well as the ability to present processed antigens to T cells. By screening a B-cell cDNA library with a DQ-beta probe, Giorda et al. (1991) Immunogenetics 33 404-408 obtained the cDNAs corresponding to the beta chains of the HLA-DQw7, -DQw8, and -DQw9 alleles. Sequence analysis revealed differences between DQw8 and DQw9 in 6 nucleotide positions that resulted in only 1 amino acid change. DQw9 encodes an aspartic acid instead of alanine at position 57. DQw7 is the same as DQw9 at this position but differs in 9 other amino acids.

Cicatricial pemphigoid (CP) is a chronic autoimmune blistering disease affecting multiple mucous membranes derived from stratified squamous epithelium and occasionally the skin. CP has a wide spectrum of disease manifestations. Patients with oral pemphigoid (OP) have a benign self-limited disease in which pathologic changes are restricted to the oral mucosa. On the other hand, patients with ocular cicatricial pemphigoid, a chronic condition marked by relapses and remissions, have ocular involvement and also perhaps involvement of other mucous membranes. All clinical types are characterized by the presence of similar anti-basement zone autoantibody. The factors that determine the development of one form of CP or the other are not known. Yunis et al. (1994) Proc. Nat. Acad. Sci. 91 7747-7751 studied class II alleles by DNA testing in 22 Caucasian patients with OP and their families (19 families). The results were compared to those obtained from 17 OCP patients and their family controls and those of 42 control Caucasian families studied for bone marrow transplantation. The results indicated that HLA-DQB1*0301 is a marker of susceptibility for both oral and ocular forms of CP. The analysis of the amino acid sequence of the DQB1 alleles present in both OP and OCP suggested that amino acid residues at position 57 and positions 71-77 may also be markers.

Delgado et al. (1996) Proc. Nat. Acad. Sci. 93: 8569-8571 compared the high-resolution typing of MHC class II loci, HLA-DRB1 and HLA-DQB1, in 21 patients with bullous pemphigoid, 17 patients with ocular cicatricial pemphigoid, and 22 patients with oral pemphigoid to a panel of 218 haplotypes of normal individuals. They found that the 3 diseases had significant association with DQB1*0301 (P=0.005, P less than 0.0001, and P=0.001 respectively). The frequencies of alleles DQB1*0302, *0303, and *06, which shared a specific amino acid sequence from position 71 to 77, were also increased (P=0.01). The findings suggested that the autoimmune response in the 3 clinically different variants of pemphigoid involves recognition by T cells of a class II region of DQB1, bound to a peptide from the basement membrane of skin., conjunctiva, or oral mucosa.

Premature ovarian failure (POF) has an autoimmune pathogenesis in a significant proportion of cases. Arif et al. (1999) J. Clin. Endocr. Metab. 84 1056-1060 found that HLA-DQB1 genotypes encoding aspartate-57 are associated with 3-beta-hydroxysteroid dehydrogenase autoimmunity in POF. Two HLA-DQB1 alleles showed positive association with 3-beta-HSD autoantibodies: *0301 and *0603, which share an asp codon at position 57. Eighteen of 21 (86%) POF patients with 3-beta-HSD autoantibodies had DQ-beta-asp57-encoding genotypes compared with 92 of 134 control subjects, and 9 of 21 (43%) cases were homozygous for codon 57 genotypes compared with 17 of 134 (13%) control subjects. These probability values were not significant after correction for multiple testing. The authors concluded that their demonstration of an association between POF, 3-beta-HSD autoimmunity, and a distinctive HLA-DQ molecule supported the hypothesis that autoantibodies to 3-beta-HSD may be markers of autoimmune ovarian failure and suggested that presentation of autoantigenic or external peptides to T lymphocytes by HLA-DQ molecules with asp57-beta chains is important in the pathogenesis of this disease.

Human Leucocyte Antigen

As used herein, the term human leucocyte antigen (HLA) has its usual meaning in the art and refers to cell surface glycoproteins encoded by closely linked genes on the short arm of chromosome 6.

The HLA type of particular interest according to the present invention is HLA class II type DQ7.

Susceptibility

According to the present invention there is provided a method of determining the susceptibility of a subject to prion disease.

The term “susceptibility” refers to the susceptibility, likelihood, vulnerability, predisposition or permissiveness of a subject to prion disease.

If a sample from a subject has HLA with DQ7 specificity, this indicates that the subject has a decreased susceptibility to prion disease: and if a sample from a subject does not have HLA with DQ7 specificity then this indicates that the subject will have an increased susceptibility to prion disease.

Sample

The “sample” may be any physical entity capable of being HLA typed for DQ7.

Preferably, the sample is or is derived from bodily fluid or tissue. More preferably, the bodily fluid is or is derived from blood—such as serum.

In a preferred embodiment, the sample comprises nucleic acid. In a highly preferred embodiment, the sample comprises nucleic acid extracted from blood.

Extraction

In a preferred embodiment of the present invention, nucleic acid—such as DNA—is isolated from blood cells by salting out as described in Miller et. Al (1988) Nuc. Acids Res. 16, 1215.

In a highly preferred embodiment of the present invention, nucleic acid—such as DNA—is isolated from blood cells using commercially available kits—such as a DNA Extraction Kit (Dynal, Merseyside, UK). In this procedure, 200 μl of anti-coagulated whole blood in a 2 ml tube is mixed with 1 ml of Red Cell Lysis Buffer 1 (5 ml concentrate+44 ml distilled water+1 ml 0.5 M EDTA pH 8) until the solution is clear. The solution is centrifuged at 10,000 rpm for 10 sec. to pellet the white blood cells and the supernatant is discarded. The tube is vortexed and 1 ml Red Cell Lysis Buffer 2 (5 ml concentrate+45 ml distilled water) is added. The solution is centrifuged for 10 sec and the supernatant discarded. 200 μl of resuspended. Dynabeads® DNA Extraction is added to the white cell pellet. The contents are immediately aspirated from the tube and transferred to a clean 2 ml tube. The tube is placed in the Dynal MPC®-Q with the magnetic bar in place and after 30 seconds the supernatant is discarded. The magnetic bar is removed and 1 ml distilled water added. The magnet is replaced and after 30 seconds, the supernatant discarded. The complex is washed a further two times and the contents of the tube containing DNA is resuspended in 200 μl distilled water.

DQ7

HLA with DQ7 specificity can be determined or typed in a sample using any suitable method. For example, serological-based methods may be used to HLA type a sample. Preferably, DNA-based methods are used to HLA type a sample.

Serological methods may be based on antigen capture by monoclonal or polyclonal antibodies and require the presence of detectable levels of HLA proteins on the surface of lymphocytes. For example, lymphocytes may be isolated by density gradient centrifugation and washed twice with a medium such as Macoy's 5a medium from 10 ml of blood mixed with 250 units of sodium heparin. B cells may then be separated using nylon-wool. T-cell depleted, B-cell enriched lymphocytes may be DQ typed by extended incubation cytotoxicity testing in accordance with Katz et al. (1987) J Periodontol 58: 607-610.

The term ‘DNA-based method’ refers to any DNA-based method that can be used to determine if a sample has HLA with DQ7 specificity. Preferably, DNA-based methods are used to determine if a sample contains a locus that infers HLA DQ7 specificity. More preferably, DNA-based methods are used for determining if a sample contains an allele that infers HLA DQ7 specificity. More preferably, DNA-based methods are used for determining if a sample contains a DQB1*030 allele—such as DQB1*03011, DQB1*03012, DQB1*0304 and/or DQB1*0309—that infers HLA DQ7 specificity.

DNA-based methods for HLA typing have been reviewed by Bidwell (1998) Immunology Today 9, 18-23 and Angelini et al. (1986) Proc. Natl. Acad. Sci. USA. 83, 4489-4493.

SSO may be performed using commercially available kits—such as the RELI SSO HLA-DQB1 Test (Dynal., Merseyside, UK).

For the RELI SSO HLA-DQB1 Test. 30 μl of 3Master Mix (Tris-HCl containing 10% glycerol, KCl, <0.001% dATP, dCTP, dGTP, dUTP, biotinylated primers, <0.01% AmpliTaq® (Taq polymerase) and 0.05% sodium azide) is aliquoted in to each PCR tube followed bat 15 μl 6 mM MgCl₂ and either 15 μl of control DNA (containing an allele such as DQB1*03011 allele), negative control (15 μl) sterile distilled water) or 15 μl of the sample to be tested. PCR amplification is performed using a thermal cycler programmed as follows: 35 cycles of 15 sec at 95° C. 45 sec at 60° C. 15 sec 72° C.: hold for 5 min at 72° C. and then 15° C. forever. When the program is finished the tubes are removed and 60 μl of Denaturation solution (3% EDRA, 1.6% NaOh and thymol blue) are added followed by 10 min incubation at room temperature. The denatured reactions may be stored at room temperature if the detection is performed within 1-2 hours. Otherwise, the denaturation reactions should be stored at 2-8° C. (for no longer then 1 week) until the detection step is performed.

For the detection step, three buffers are required: (1) Working Hybridisation Buffer prepared by mixing 55 ml SSPE Concentrate (Sodium phosphate solution with NaCl. EDTA and 1% Proclin 150®). 213 ml distilled water and 6.9 ml SDS Concentrate (SDS with 1% Proclin 150®); (2) Working Wash Buffer prepared by mixing 65 ml SSPE Concentrate, 1228.5 ml distilled water and 6.5 ml SDS Concentrate. 275 ml is used for Stringent Wash Buffer and 1025 ml is used for Ambient Wash Buffer; (3) Working Citrate Buffer prepared by diluting 30 ml of Citrate Concentrate (sodium citrate solution) with 570 ml distilled water.

The Hybridisation buffer and Stringent Wash buffer are warmed to 50° C. prior to use and a water bath set to the same temperate. An HLA-DQB1 typing strip is placed in each well of a Typing Tray. 5 ml of pre-warmed Hybridisation Buffer is added to each well followed by 70 μl of denatured amplified samples or controls to each well. A lid is placed on the Typing Tray and the tray incubated at 50° C. for 30 min at 60 rpm. The contents of each of the wells are removed and 5 ml of Ambient Wash Buffer added. The contents of each of the wells are removed and 5 ml Stringent Wash Buffer is added for 15 min at 50° C. at 60 rpm. A Working Conjugate Solution is prepared by adding 5.3 ml of Ambient Wash Buffer to 16 μl Streptavidin-HRP Conjugate (Streptavidin-horseradish peroxidase conjugate in an ACES solution with NaCl and 1% Proclin 150®) for each strip being assayed. The tray is removed from the water bath and the contents removed from each of the wells. 5 ml of Working Conjugate Solution is added to each well and the trace mixed for 15 min at 60 rpm. The contents are removed from the wells and 5 ml Ambient Wash Buffer added to each well for 5 min at 60 rpm. The Ambient Wash Buffer step is repeated once. The contents are removed from the wells and 5 ml Citrate Buffer added for 5 min at 60 rpm. A Working Substrate (mixture of Substrate A (citrate solution containing 0.01% H₂O₂ and 0.1% ProClin 150®) and Substrate B (0.1% 3,3′,5,5′-tetramethylbenzidine in 40% dimethylfonnamide) is prepared by calculating the amount of Substrate A needed by using the calculation [4.4 ml×the number of strips]. The amount of Substrate B needed is determined by the calculation [1.1 ml×the number of strips]. The contents of the wells are removed and 5 ml Working Substrate added for 10 min at 60 rpm. The contents of the wells are removed and 5 ml distilled water added for 5 min at 60 rpm—this step is repeated twice. 5 ml Citrate Buffer is added to each well and strips interpreted manually using the HLA-DQB1 Overlay and Score Sheet included with the kit. For each blue line that has an intensity greater than the control line a positive signal is recorded. The pattern is compared with the Dynal RELI SSO HLA-DQB Interpretation Table included with the kit.

A positive result is obtained when a sample contains a DQB1*030 allele—such as DQB1*03011, DQB1*03012, DQB1*0304 and/or DQB1*0309. The presence of one or more of these alleles infers HLA of DQ7 type. Specifically, a positive result for HLA with DQ7 specificity is obtained when blue lines with an intensity greater than the control line are present in, for example, lane 7, lane 1, lane 17, lanes 22 and 23 (DQB1*03011, DQB1*03012 and DQB1*0309) and/or lane 25 and/or lane 7, lane 11, lane 16, lanes 22-23 and lane 25 (DQB1*0304).

SSP may be performed according to Bunce et al. (1995) Tissue Antigens 46, 355-367 or using commercially available kits—such as the AllSet SSP DQB1*03 Test (Dynal, Merseyside, UK).

Purified DNA may be prepared using the previously mentioned methods such as the DNA Extraction Kit (Dynal, Merseyside, UK).

5 μl) of the supplied primer solutions are distributed to each PCR tube. To perform one DQB1*03 typing (11 PCR reactions) the following PCR mix is made: 112 μl Dynal AllSet SSP Master Mix (Tris-HCl, KCl, Gelatin, MgCl2, dATP, dCTP, dGTP, dTTP, glycerol and cresol red); 26.6 μl sample DNA (50 ng/μl); 1.12 μl Taq DNA polymerase. The contents are mixed and 10 μl of the PCR mix is dispensed into each well. The PCR cycling parameters used are: 94° C. for 2 min. 10 cycles of 94° C. for 10 sec and 65° C. for 60 sec; 20 cycles of 94° C. for 10 sec; 61° C. for 50 sec and 72° C. for 30 sec.

A positive result is obtained when a sample contains a DQB1*030 allele—such as DQB1*03011, DQB1*03012, DQB1*0304 and/or DQB1*0309—that infers HLA DQ7 specificity. Accordingly, HLA with DQ7 specificity is identified when a positive signal is obtained, for example, in tube 1 (PCR product 140 bp) and tube 2 (PCR product 125 bp) (DQB1*0301 and DQB1*0302) and/or tube 1 and tube 5 (130 bp) (DQB1*0304) and/or tube 1, tube 2, and tube 10 (PCR product 120 bp) (DQB1*0309).

In another embodiment of the present invention, the prion susceptibility of mammals such as livestock mammals eg. sheep and/or cows may be determined. In this embodiment, the DNA-based method is preferably DNA sequencing. DNA sequencing may be used to identify one or more nucleotide and/or amino acid sequences—such as one or more loci and/or one or more alleles—that infer the equivalent HLA DQ7 specificity in MHC of livestock mammals. For example, the sequence of DQB1*030 alleles that infer HLA DQ7 specificity are publicly available in databases, for example, the 5′ flanking region of the DQB1*0301 allele from humans (accession number AF217420); the first domain, exon 2 of the DQB1*0304 allele from humans (accession number M74842). Databases of nucleotide and amino acid sequences from livestock can be searched to identify sequences that are homologous to those sequences that infer HLA DQ7 specificity. Accordingly, it will be appreciated by a person skilled in the art that the methods of the present invention max also be used for determining the susceptibility of livestock to prion disease by determining the equivalent HLA DQ7 specificity.

It will be apparent to a person skilled in the art that the methods of the present invention may be combined with other methods that determine the susceptibility of a subject to prion disease (such as PrP Met 129 typing, or further combinations of blood-based markers of which DQ7 is a preferred example) thereby advantageously providing a second readout for each subject. In one such method, the genotype of PrP 129 is determined as described in WO 98/16834. Collinge et al. (1991) Lancet 337, 1441-1442, Palmer et al. (1991) Nature 352, 340-342 and Collinge et al. (1996) Lancet 348, 56. A polymorphism at amino acid 129 of PrP has been identified (with either methionine or valine) which contributes to the genetic susceptibility to sporadic and acquired human prion disease—of a panel of patients with vCJD, the great majority are homozygotes for methionine at polymorphic residue 129. Thus, a subject having PrP Met 129 and also lacking HLA type DQ7 might be expected to have the highest susceptibility to prion disease and vice versa. The combination of methods may provide a more robust indicator of the susceptibility of a subject to prion disease.

As used herein the term “livestock” refers to any farmed animal. Preferably, livestock are one or more of a pig, sheep, cow or bull. More preferably, livestock are a cow, bull or sheep.

Nucleotide Sequence

Aspects of the present invention may involve the use of nucleotide sequences, which are available in databases.

As used herein, the term “nucleotide sequence” is synonymous with the term “polynucleotide”.

The nucleotide sequence may be DNA or RNA of genomic or synthetic or recombinant origin. The nucleotide sequence may be double-stranded or single-stranded whether representing the sense or antisense strand or combinations thereof.

General Recombinant DNA Methodology Techniques

Although in general the techniques mentioned herein are well known in the art, reference may be made in particular to Sambrook et al., Molecular Cloning, A Laboratory Manual (1989) and Ausubel et al., Short Protocols in Molecular Biologic (1999) 4^{th} Ed. John Wiley & Sons. Inc.

In another aspect, the present invention relates to the breeding of mammals—such as livestock—with a decreased susceptibility to prion disease. Livestock that have one or more MHC molecules with equivalent HLA DQ7 specificity, may be bred. Those offspring that have one or more MHC molecules with equivalent HLA DQ7 specificity may be selected and used in further breeding programs. Various methods of breeding may be used including in-breeding, out-breeding or any other breeding method known to a person skilled in the art.

In a further aspect, the present invention relates to transgenic mammals—such as livestock—which have one or more nucleotide and/or amino acid sequences with equivalent HLA DQ7 specificity inserted in to their genome. Thus, the transgenic mammals may have a decreased susceptibility to prion disease. MHC molecules that infer equivalent HLA DQ7 specificity—such as one ore more loci and/or one or more alleles—may be introduced into mammals using several techniques well known to a person skilled in the art, such as: (1) the transfer of cells from one embryo to another; (2) introduction of cells infected with a retrovirus; (3) microinjection of cDNA into the pronucleus of a fertilised egg according to the methods described by Scott et al. (1989), Cell 59, 847-857 and Scott et al. (1992) Protein Sci., 1, 986-997; (4) implantation of multiple eggs into a single animal. Known procedures are then used to determine whether the resulting offspring are transgenic mammals.

In yet a further aspect, the present invention may also relate to transgenic mammals—such as transgenic mice—which have one or more have one or more MHC molecules that infer equivalent HLA DQ7 specificity—such as one or more loci and/or one or more alleles—deleted from their genome such that the transgenic mammals have an increased susceptibility to prion disease.

### EXAMPLES

The present invention is illustrated with reference to the following examples.

### Example 1

Identification of HLA with DQ7 specificity that is associated with susceptibility to prion disease.

DNA is isolated from 49 human patients infected with vCJD and 197 control human patients using a DNA Extraction Kit (Dynal, Merseyside, UK; Product Number 633.XX). 200 μl of anti-coagulated whole blood in a 2 ml tube from each patient is mixed with 1 ml of Red Cell Lysis Buffer 1 (5 ml concentrate+44 ml distilled water+1 ml 0.5 M EDTA pH 8) until the solution is clear. The solution is centrifuged at 10,000 rpm for 10 sec. to pellet the white blood cells and the supernatant is discarded. The tube is vortexed and 1 ml Red Cell Lysis Buffer 2 (5 ml concentrate+45 ml distilled water) added. The solution is centrifuged for 10 sec and the supernatant discarded. 200 μl of resuspended Dynabeads® DNA Extraction is added to the white cell pellet and the contents of the tube immediately aspirated and transferred to a clean 2 ml tube. The tube is placed in a Dynal.MPC®-Q with the magnetic bar in place and after 30 seconds the supernatant is discarded. The magnetic bar is removed and 1 ml distilled water added. The magnet is replaced and after 30 seconds, the supernatant discarded. The complex is washed a further two times and the DNA extracted from the 246 blood samples is resuspended in 200 μl distilled water.

HLA typing is performed using the RELI SSO HLA-DQB1 Test (Dynal, Merseyside, UK: Product Number 820.XX).

30 μl of Master Mix (Tris-HCl containing 10% glycerol, KCl, <0.00% dATP, dCTP, dGTP, dUTP, biotinylated primers, <0.01% AmpliTaq® (Taq polymerase) and 0.05% sodium azide) is aliquoted in to each PCR tube followed by 15 μl of 6 mM MgCl₂ and either 15 μl of control DNA (containing a DQB1*030 allele), negative control (sterile distilled water) or DNA extracted from each of the 246 blood samples (using the above-mentioned DNA Extraction Kit). PCR amplification is performed using a thermal cycler programmed as follows: 35 cycles of 15 sec at 95° C., 45 sec at 60° C., 15 sec 72° C.; hold for 5 min at 72° C. and then 15° C. forever. When the program is finished the tubes are removed and 60 μl of Denaturation solution (3% EDTA. 1.6% NaOH and thymol blue) are added followed by 10 min incubation at room temperature.

For the detection step, three buffers are used: (1) Working Hybridisation Buffer prepared by mixing 55 ml SSPE Concentrate (Sodium phosphate solution with NaCl, EDTA and 1% Proclin 150®), 213 ml distilled water and 6.9 ml SDS Concentrate (SDS with 1% Proclin 150®); (2) Working Wash Buffer prepared by mixing 65 ml SSPE Concentrate. 1228.5 ml distilled water and 6.5 ml SDS Concentrate. 275 ml is used for Stringent Wash Buffer and 1025 ml is used for Ambient Wash Buffer; (3) Working Citrate Buffer prepared by diluting 30 ml of Citrate Concentrate (sodium citrate solution) with 570 ml distilled water.

The Hybridisation buffer and Stringent Wash buffer are warmed to 50° C. prior to use and a water bath set to the same temperate. An HLA-DQB1 typing strip is placed in each well of a Typing Tray. 5 ml of prewarmed Hybridisation Buffer is added to each well followed by 70 μl of denatured amplified samples or controls to each well. A lid is placed on the Typing Tray and the tray incubated at 50° C. for 30 min at 60 rpm. The contents of each of the wells are removed and 5 ml of Ambient Wash Buffer added. The contents of each of the wells are removed and 5 ml Stringent Wash Buffer is added for 15 min at 50° C. at 60 rpm. A Working Conjugate Solution is prepared by adding 5.3 ml of Ambient Wash Buffer to 16 μl Streptavadin-HRP Conjugate (Streptavadin-horseradish peroxidase conjugate in an ACES solution with NaCl and 1% Proclin 150®) for each strip being assayed. The tray is removed from the water bath and the contents removed from each of the wells. 5 ml of Working Conjugate Solution is added to each well and the tray mixed for 15 min at 60 rpm. The contents are removed from the wells and 5 ml Ambient Wash Buffer added to each well for 5 min at 60 rpm. The Ambient Wash Buffer step is repeated once. The contents are removed from the wells and 5 ml Citrate Buffer added for 5 min at 60 rpm. A Working Substrate (mixture of Substrate A (citrate solution containing 0.01% H₂O₂ and 0.1% ProClin 150®) and Substrate B (0.1% 3,3′,5,5′-tetramethylbenzidine in 40% dimethylformamide)) is prepared by calculating the amount of Substrate A needed by using the calculation [4.4 ml×the number of strips]. The amount of Substrate B needed is determined by the calculation [1.1 ml×the number of strips]. The contents of the wells are removed and 5 ml Working Substrate added for 10 min at 60 rpm. The contents of the wells are removed and 5 ml distilled water added for 5 min at 60 rpm—this step is repeated twice. 5 ml citrate buffer is added to each well.

The strips are interpreted manually using the HLA-DQB1 Overlay and Score Sheet included with the kit. For each blue line that has an intensity greater than the control line a positive signal is recorded. The pattern is compared with the Dynal RELI SSO HLA-DQB Interpretation Table included with the kit.

The following results are obtained:

| | vCJD infected patients | Control patients |
|---|---|---|
| Specificity | Total patients = 49 | Total patients = 197 |
| DQ2 | 40% | 40.6% |
| DQ4 | 4% | 2.5% |
| DQ5 | 32% | 26.9% |
| DQ6 | 54% | 41.6% |
| DQ7 | 12.2% | 35.5% |
| DQ8 | 18% | 19.8% |
| DQ9 | 18% | 12.7% |

By comparing the results of the HLA DQ7 specificity between the vCJD infected patients (12.2%) and the control patients (35.5%) HLA with DQ7 specificity is lower in patients that have vCJD. Accordingly, the presence of HLA with DQ7 specificity in subjects with vCJD is lower than in control patients.

Thus, it is demonstrated that the presence of HLA with DQ7 specificity in a subject is associated with a decreased susceptibility to prion disease; the absence of HLA with DQ7 specificity in a subject is associated with an increased susceptibility to prion disease.

### Example 2

Determining the susceptibility of a subject to prion disease using a DNA-based method (SSO).

DNA Extraction and HLA typing are performed as described in Example 1, on a sample derived from a human patient.

The following control reactions are used: (1) Positive control=DNA extracted from a patient that has vCJD; (2) Negative control=distilled water. The HLA-DQB1 typing strips are interpreted manually using the HLA-DQB1 Overlay and Score Sheet included with the kit. For each blue line that has an intensity greater than the control line a positive signal is recorded. The pattern is compared with the Dynal RELI SSO HLA-DQB Interpretation Table included with the kit.

For the human patient being tested positive signals (i.e. blue lines with an intensity greater than the control line) are present in lane 7, lane 11, lane 17, lanes 22-23 and lane 25. Thus, the subject has DQB1*0301 (DQB1*03011/DQB1*03012) and DQB1*0309 and therefore has HLA with DQ7 specificity.

For the positive control positive signals (i.e. blue lines with an intensity greater than the control line) are present in lane 7, lane 11, lane 17, lanes 22-23 and lane 25. Thus, the control has DQB1*0301 (DQB1*03011/DQB1*03012) and DQB1*0309 which infers HLA DQ7 specificity.

For the negative control, no positive signals are obtained.

Thus, it is demonstrated that the human patient has HLA with DQ7 specificity i.e. the human patient has a decreased susceptibility to vCJD.

### Example 3

Determining the susceptibility of a subject to prion disease using a DNA-based method (SSO).

DNA Extraction and HLA typing are performed as described in Example 1 on a human patient. The controls used are the same as Example 2.

The HLA-DQB1 typing strips are interpreted manually using the HLA-DQB1 Overlay and Score Sheet included with the kit. For each blue line that has an intensity greater than the control line a positive signal is recorded. The pattern is compared with the Dynal RELI SSO HLA-DQB Interpretation Table included with the kit. Positive signals (i.e. blue lines with an intensity greater than the control line) are not obtained for DQB1*030 alleles that infer HLA DQ7 specificity.

The results for the positive and negative controls are the same as Example 2.

Thus, it is demonstrated that the human patient does not have HLA with DQ7 specificity i.e. the human patient has an increased susceptibility to vCJD.

### Example 4

Determining the susceptibility of a subject prion to disease using a DNA-based method (SSP).

DNA extraction is performed according to Example 1, on a human patient. The controls used are the same as Example 2.

The AllSet SSP DQB1*03 Test (Dynal, Merseyside, UK; Product No. 581.XX) is used. 5 μl of the supplied primer solutions are distributed to each PCR tube. To perform one DQB1*03 typing (11 PCR reactions) the following PCR mix is made: 112 μl Dynal AllSet SSP Master Mix (Tris-HCl, KCl, Gelatin, MgCl₂, dATP, dCTP, dGTP, dTTP, glycerol and cresol red). 26.6 μl sample DNA (50 ng/[l); 1.12 μl Taq DNA polymerase. The contents are mixed and 10 μl of the PCR mix is dispensed into each well. The PCR cycling parameters used are: 94° C. for 2 min; 10 cycles of 94° C. for 10 sec and 65° C. for 60 sec; 20 cycles of 94° C. for 10 sec, 61° C. for 50 sec and 72° C. for 30 sec.

A positive signal is obtained for tube 1 (PCR product 140 bp) and tube 2 (PCR product 125 bp) i.e. a positive result for DQB1*0301 (DQB1*03011 and DQB1*03012) which infers HLA DQ7 specificity.

For the positive control, positive signals are obtained for tube 1 (PCR product 140 bp) and tube 2 (PCR product 125 bp) i.e. a positive result for DQB1*0301 (DQB1*03011 and DQB1*03012) which infers HLA DQ7 specificity.

For the negative control, no positive signals are obtained.

Thus, it is demonstrated that the human patient has HLA with DQ7 specificity i.e. the human patient has a decreased susceptibility to vCJD.

### Example 5

Determining the susceptibility of a subject prion to disease using a DNA-based method (SSP).

DNA extraction is performed according to Example 1 and HLA typing is performed according to Example 3, on a human patient. The controls used are the same as Example 4.

A positive signal is not obtained for any DQB1*030 alleles that infer HLA DQ7 specificity.

The results for the controls are the same as Example 4.

Thus, it is demonstrated that the human patient does not have HLA with DQ7 specificity i.e. the patient has an increased susceptibility to vCJD.

### Example 6

This example illustrates a significantly reduced frequency of the HLA class II type DQ7 (DQB1*0301/4/9) in patients with vCJD, whereas the incidence in sporadic CJD was similar to that of the control group.

Without wishing to be bound by theory, this molecular marker may be itself directly involved with vCJD susceptibility or a tightly linked gene may be responsible for this effect.

This example presents a study to determine the HLA type of vCJD patients for comparison with normal controls and sporadic CJD. The aetiology of sporadic CJD, the main differential diagnosis of vCJD, remains unclear but its random worldwide distribution and lack of clustering or association with local scrapie prevalence argues against an acquired aetiology. Somatic mutation of the prion protein gene, or spontaneous formation of PrP^{Sc}, are thought more likely causes.

An initial study was performed on 32 DNA samples extracted from vCJD patient tissue and compared with samples from 12 cases of sporadic CJD. Tissue samples were obtained at autopsy with consent of relatives. These samples were HLA tipped for HLA-A, B, C, DRB and DQB1 at low/medium resolution using a sequence-specific-primer polymerase chain reaction (PCR-SSP) methodology (Bunce et al). All of the samples used in the study were confirmed as being from British Caucasoids. HLA frequencies from a panel of 197 consecutive cadaveric organ donors were used as control values.

The only statistically significant deviation from control frequencies was seen at the DQB1 locus only 2 out of the 32 vCJD patients were positive for DQB1*0301/4/9 (DQ7), compared with 70 out of 197 controls (35.5%) with a two-sided Fisher's exact test reporting an uncorrected p value of 0.0004. In the case of sporadic CJD, 50% of patients were DQ7 (6/12) which was not significantly different from the controls (p=0.36). Following this finding an additional 18 vCJD and 14 sporadic CJD patients were typed for HLA-DQB1 only using a commercial PCR-SSOP kit (Reli™, Dynal UK). Table 1 shows the percent phenotype frequencies of HLA-DQ types in 50 vCJD patients, 26 sporadic CJD patients and 197 controls. HLA-DQ types were assigned by considering the DQB1 alleles of each subject. The frequency of HLA-DQ7 remained significantly under-represented (p=0.0010) in the vCJD patients, with only 6 out of 50 having this phenotype. HLA-DQ frequencies in the sporadic CJD group remained comparable with those of the controls.

Currently all victims of vCJD studied have been homozygous for methionine with respect to a common polymorphism in the PRNP gene. Around 38% of the normal UK population have this genotype. Our data suggest that the presence of DQ7 is somehow protective with a relative risk for individuals negative for DQ7 of 3.3.

Thus, it is demonstrated that DQ7 typing suspected vCJD patients in conjunction with PRNP genotyping is a useful tool in diagnosis.

While a firm tissue-based diagnosis of vCJD can be achieved by tonsil biopsy, clearly blood-based diagnostics according to the present invention are preferable.

Without wishing to be bound by theory, the molecular basis for the protective association of DQ7 and vCJD is unclear. It is possible that MHC class II molecules themselves play a direct role in disease pathogenesis or that a gene linked to the DQB locus is involved. A possible role for MHC class II mall be in fortuitous carriage of PrP^{Sc} around the body or indeed from the gut into the lymphoreticular system, a role for which the DQ7 molecule is less efficient. Alternatively the HLA-DQ7 molecules may be more efficient at presenting a putative pathogenic peptide and in this way initiate an immune response. Working of this invention may reveal a crucial role for MHC Class II molecules in disease pathogenesis. Thus, a target for therapeutic intervention is disclosed.

TABLE 1

| | | Sporadic | Consecutive |
|---|---|---|---|
| | vCJD | CJD | cadaveric controls |
| DQ Type | (50 patients) | (26 patients) | (n = 197) |
| DQB1*02 - DQ2 | 42% (21) | 30.8% (8) | 40.6% (80) |
| DQB1*04 - DQ4 | 4% (2) | 3.8% (1) | 2.5% (5) |
| DQB1*05 - DQ5 | 32% (16) | 30.8% (8) | 26.9% (53) |
| DQB1*06 - DQ6 | 58% (29) | 38.5% (10) | 41.6% (82) |
| DQB1*0301/4/9 - DQ7 | 12% (6) | 46.2% (12) | 35.5% (70) |
| | p = 0.0010* | | |
| DQB1*0302/5/7/8 - DQ8 | 18% (9) | 15.4% (4) | 19.8% (39) |
| DQB1*0303/6 - DQ9 | 18% (9) | 15.4% (4) | 12.7% (25) |

*Uncorrected p value from a two-sided Fisher's exact test reports an extremely significant deviation from the control frequency.

References

Aguzzi, A.; Brandner, S. Lancet 354: 22-25, 1999.

Aguzzi, A.; Weissmann, C. A suspicious signature. Nature 383: 666-667, 1996.

Basler, K. et al., Cell 46: 417-428, 1986.

Bertoni, J. M. . et al., J.A.M.A. 268: 2413-2415, 1992.

Beyreuther, K.; Masters, C. L. Nature 370: 419-420, 1994.

Bosque, P. J. et al., Neurology 42: 1864-1870, 1992.

Brown, P. et al., J. Neurol. Sci. 112: 65-67, 1992.

Brown, P. et al., Lancet 337: 1019-1022, 1991.

Brown, P. et al., Ann. Neurol. 31: 282-285, 1992.

Brown, P. et al., Neurology 42: 422-427, 1992.

Bueler, H. et al.. Cell 73: 1339-1347, 1993.

Bueler, H. et al., Molec. Med. 1: 19-30, 1994.

Bunce M, O'Neill C M, Barnardo M C et al. Tissue Antigens 1995;46: 355-67.

Campbell, T. A. et al., Neurology 46: 761-766, 1996.

Carlson, G. A. et al., Cell 46: 503-511, 1986.

Chapman, J. et al., Neurology, 46: 758-761, 1996.

Chapman, J. et al., Neurology 44: 1683-1686, 1994.

Chapman, J. et al., Neurology 42: 1249-1250, 1992.

Chapman, J.; Korczyn, A. D. Neuroepidemiology 10: 228-231, 1991.

Chiesa, R. et al., Proc. Nat. Acad. Sci. 97: 5574-5579, 1960.

Chiesa, R.; Piccardo. P.; Ghetti, B.; Harris, D. : Neuron 21: 1339-1351, 1998.

Collinge, J. Hum. Mol. Genet. 6: 1699-1705, 1997.

Collinge, J. et al., Brain 115: 687-710, 1992.

Collinge, J. et al., Lancet II: 15-17, 1989.

Collinge, J. et al., Lancet 336: 7-9, 1990.

Collinge, J. Palmer, M. S. Dryden, A. J. Lancet 337: 1441-1442, 1991.

Collinge, J. et al., Am. J. Hum. Genet. 49: 1351-1354, 1991.

Collinge J. Variant Creutzfeldt-Jakob disease, Lancet 1999:354: 317-23.

Collinge, J. et al., Nature 383: 685-690, 1996.

Collinge. J. et al., Nature 370: 295-297, 1994.

Colombo, R. et al., Am. J. Hum. Genet. 67: 528-531, 1960.

de Silva, R. et al., Neurosci. Lett. 179: 50-52, 1994.

Deslys, J.-P. et al., Lancet 351: 1251 only, 1998.

Dlouhy, S. R. et al., Nature Genet. 1: 64-67, 1992.

Doh-ura, K. et al., Nature 353: 801-802, 1991.

Doh-ura, K. et al., Biochem. Biophys. Res. Commun. 163: 974-979, 1989.

Duchen, L. W.; Poulter, M.; Harding, A. E.: Brain 116: 555-567, 1993.

Farlow, M. R. Neurology 39: 1446-1452, 1989.

Forloni, G.; et al., Nature 362: 543-546, 1993.

Gabizon, R et al., Am. J. Hum. Genet. 53: 828-835, 1993.

Gajdusek, D. C. Europ. J. Epidemiol. 7: 567-577, 1991.

Gambetti, P et al., Brit. Med. Bull. 49: 980-994, 1993.

Ghetti, B et al., Neurology, 39: 1453-1461, 1989.

Giaccone, G et al., Proc. Nat. Acad. Sci. 89: 9349-9353, 1992.

Goldfarb, L. G et al., Molec. Neurobiol. 8: 89-97, 1994.

Goldfarb. L. G. et al., Exp. Neurol. 108: 247-250, 1990.

Goldfarb, L. G. et al., Ann. Neurol. 31: 274-281, 1992.

Goldfarb, L. G. et al., Proc. Nat. Acad. Sci. 88: 10926-10930, 1991.

Chapman, J. et al., Europ. J. Epidemiol, 7: 477-486, 1999.

Goldfarb, L. G. et al., Lancet 337: 425, 1991.

Goldfarb, L. G. et al., Lancet 336: 514-515, 1990.

Goldfarb, L. G. et al.,. Science 258: 806-808, 1992.

Goldgaber, D. et al., Exp. Neurol. 106: 204-206, 1989.

Goldhammer, Y. et al., Neurology 43: 2718-2719, 1993.

Griffith, J. S.: Self-replication and scrapie. Nature 215: 1043-1044, 1967.

Haltia, M. et al., Europ. J. Epidemiol. 7: 494-500, 1991.

Hegde, R. S. et al., Science 279:827-834, 1998.

Hegde, R. S. et al., Nature 402: 732-736, 1999.

Hill A F, Butterworth R J, Joiner S et al. Lancet 1999:353: 183-9.

Horwich, A. L.; Weissman, J. S. Cell 89: 499-510, 1997.

Hsiao, K. et al., Nature 338: 342-345, 1989.

Hsiao, K et al., Neurobiol. Aging 11: 302, 1990.

Hsiao, K et al.,. Nature Genet. 1: 68-71, 1992.

Hsiao, K et al., New Eng. J. Med. 324: 1091-1097, 1991.

Hsiao, K. K et al., Proc. Nat. Acad. Sci. 91: 9126-9130, 1994.

Ironside. J. W et al.,. Cold Spring Harbor Symp. Quant. Biol. 61: 523-530, 1996.

Jendroska, K et al., J. Neurol. Neurosurg. Psychiat. 57: 1249-1251, 1994.

Kitamoto, T et al., Biochem. Biophys. Res. Commun. 191: 709-714, 1993.

Kocisko, D. A et al., Nature 370: 471-474, 1994.

Krasemann, S et al., Molec. Brain Res. 34: 173-176, 1995.

Kretzschmar, H. A et al., Acta Neuropath. 89: 96-98, 1995.

Kretzschmar, H. A et al., DNA 5: 315-324, 1986.

Kuwahara, C. et al., Nature 400: 225-226, 1999.

Laplanche, J.-L et al., Brain 122: 2375-2386, 1999.

Laplanche, J. L et al., Ann. Neurol. 31: 345, 1992.

Lee, H. S et al., Am. J. Hum. Genet. 64: 1063-1070, 1999.

Liao, Y.-C. J. et al., Science 233: 364-367, 1986.

Lindquist, S. Cell 89: 495-498, 1997.

Little, B. W et al. Ann. Neurol. 20: 231-239, 1986.

Lugaresi, E.; et al., New Eng. J. Med. 315: 997-1003, 1986.

Lugaresi, E et al., Rev. Neurol. 142: 791-792, 1986.

Mallucci, G. R et al., Brain 122: 1823-1837, 1999.

Manetto, V et al., Neurology 42: 312-319, 1992.

Manson, J. C et al., Neurodegeneration 3:331-340, 1994.

Manuelidis, L et al., EMBO J. 6: 341-347, 1987.

Mastrianni, J. A et al., Neurology 45: 2042-2050, 1995.

Medori, R. et al., Neurology 42: 669-670, 1992.

Medori, R et al., Am. J. Hum. Genet. 53: 822-827, 1993.

Medori, R et al., New Eng. J. Med. 326: 444-449, 1992.

Meggendorfer, F. Z. Ges. Neurol. Psychiat. 128: 337-341, 1930.

Meiner, Z.; Gabizon, R.; Prusiner, S. B. Medicine 76: 227-237, 1997.

Mestel, R. : Putting prions to the test. Science 273: 184-189, 1996.

Mitrova, E.; Lowenthal, A.; Appeal. B. Europ. J. Epidemiol. 6: 233-238, 1990.

Monari, L. et al.,. Proc. Nat. Acad. Sci. 91: 2839-2842, 1994.

Montrasio, F et al., Science 288: 1257-1259, 1960.

Montrasio F. Frigg R, Glatzel M et al. Science 2000;288: 1257-9.

Mouillet-Richard et al., Science 289: 1925-1928, 1960.

Mouillet-Richard, S. et al., J. Neurol. Sci. 168: 141-144, 1999.

Nieto, A et al., Lancet 337: 622-623, 1991.

Oesch, B et al.,. Cell 40: 735-746, 1985.

Owen, F et al., Nucleic Acids Res. 18: 3103, 1990.

Owen, F et al., Molec. Brain Res. 13: 155-157, 1992.

Owen, F et al., Lancet I: 51-52, 1989.

Owen. F et al., Molec. Brain Res. 7: 273-276, 1990.

Pablos-Mendez, et al., Lancet 341: 159-161, 1993.

Palmer, M. S.; Collinge, J. Hum. Mutat. 2:168-173, 1993.

Palmer, M. S. et al., Nature 352: 340-342, 1991. Erratum: Nature 352: 547, 1991.

Perry, R. T. et al., Am. J. Meed. Genet. 60: 12-18, 1995.

Pocchiari, M et al., Ann. Neurol. 34: 802-807, 1993.

Poulter, M et al., Brain 115: 675-685, 1992.

Prusiner, S. B. Annu. Rev. Med. 38: 381-398, 1987.

Prusiner, S. B.: Molecular biology of prion diseases. Science 252: 1515-1522, 1991.

Prusiner, S. B. Ann. Rev. Microbiol. 48: 655-686, 1994.

Prusiner, S. B. Science 216: 136-144, 1982.

Prusiner, S. B. Cold Spring Harbor Symp. Quant. Biol. 61: 473-493, 1996.

Puckett, C et al.,. Am. J. Hum. Genet. 49: 320-329, 1991.

Reder, A. T et al., Neurology 45: 1068-1075, 1995.

Riek, R et al., Proc. Nat. Acad. Sci. 95: 11667-11672, 1998.

Rivera, H et al.,. J. Med. Genet. 26: 626-630, 1989.

Robakis, N. K et al., Biochem. Biophys. Res. Commun. 140: 758-765, 1986.

Sailer, A et al., Cell 77: 967-968, 1994.

Sakaguchi, S et al., Nature 380: 528-531, 1996.

Samaia, H. B et al., Nature 390: 241 only, 1997.

Schellenberg, G. D. et al., Am. J. Hum. Genet.49: 511-517, 1991.

Schnittger, S et al., Genomics 14: 740-744, 1992.

Scott, M et al., Cell 59: 847-857, 1989.

Shibuya, S et al., Lancet 351: 419 only, 1998.

Shmerling, D et al., Cell 93: 203-214, 1998.

Simon, E. S et al., Ann. Neurol. 47: 257-260, 1960.

Sparkes R. S et al., Proc. Nat. Acad. Sci. 83: 7358-7362, 1986.

Speer, M. C el al., Genomics 9: 366-368, 1991.

Supattapone, S et al., Cell 96: 869-878, 1999.

Tagliavini, F et al., EMBO J. 10: 513-519, 1999.

Tagliavini, F et al., Cell 79: 695-703, 1994.

Tateishi, J. et al., Nature 376: 434-435, 1995.

Telling, G. C et al., Science 274: 2079-1962, 1996.

Telling, G. C et al., Cell 83: 79-90, 1995.

Ter-Avanesyan, M. D. et al., Genetics 137: 671-676, 1994.

Tobler, I et al., Nature 380: 639-642, 1996.

Wadsworth J D F, Joiner S, Hill A F, et al. Lancet 2001; 358: 171-180.

Westaway, D et al., Cell 76: 117-129, 1994.

Whittington, M. A et al., Nature Genet. 9: 197-201, 1995.

Wickner, R. B. Science 264: 566-569, 1994.

Windl, O et al., Hum. Genet. 105: 244-952, 1999.

Yamada, M et al., Neurology 43: 2723-2724, 1993.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method of determining the susceptibility of a subject to a prion disease comprising the steps of:
(a) providing a sample from said subject; and
(b) determining the human leucocyte antigen specificity of said sample, wherein
if said sample has DQ7 human leukocyte antigen specificity, then said subject has a decreased susceptibility to prion disease; and
if said sample does not have DQ7 human leucocyte antigen specificity, then said subject has an increased susceptibility to prion disease.

2. A method according to claim 1 wherein the human leucocyte antigen specificity is determined by nucleic acid-based methods.

3. A method according to claim 2, wherein the nucleic acid-based methods comprise sequence specific primer polymerase chain reaction (PCR-SSP).

4. A method according to claim 1, wherein the prion disease is vCJD.

5. A method according to claim 1, wherein the sample is or is derived from bodily fluid or tissue.

6. A method according to claim 5, wherein said bodily fluid or tissue is blood.

7. A method according to claim 5 wherein the sample is or is derived from nucleic acid extract from the bodily fluid or tissue.

8. A method according to claim 2, wherein the prion disease is vCJD.

9. A method according to claim 3, wherein the prion disease is vCJD.

10. A method according to claim 2, wherein the sample is or is derived from bodily fluid or tissue.

11. A method according to claim 3, wherein the sample is or is derived from bodily fluid or tissue.

12. A method according to claim 4, wherein the sample is or is derived from bodily fluid or tissue.

13. A method according to claim 5, wherein the sample is or is derived from bodily fluid or tissue.

14. A method according to claim 6 wherein the sample is or is derived from nucleic acid extract from the bodily fluid or tissue.
